(19) United States
(12) Patent Application Publication
(10) Pub. No.: US 2025/0192472 A1
(43) Pub. Date: Jun. 12, 2025

(54) ELECTRICAL CONNECTOR

(71) Applicant: St. Jude Medical, Cardiology Division, Inc., St. Paul, MN (US)

(72) Inventors: Isaac Remer, Richfield, MN (US); Jodee Wakefield, Golden Valley, MN (US); Zach Helgeson, Richfield, MN (US); Timothy S. Marass, Minneapolis, MN (US); Frank Carlson, St. Paul, MN (US)

(21) Appl. No.: 18/845,735

(22) PCT Filed: Mar. 27, 2023
(86) PCT No.: PCT/US2023/064991

§ 371 Date: Sep. 10, 2024

Related U.S. Application Data
(60) Provisional application No. 63/324,352, filed on Mar. 28, 2022.

(51) Int. Cl. H01R 13/52; A61B 18/00; A61B 18/14; H01R 12/77

(52) U.S. Cl. CPC H01R 13/521; A61B 18/1492; H01R 12/777; H01R 13/5205; A61B 2018/00178; H01R 2201/12

(57) ABSTRACT

An electrical connector for a catheter includes a plug portion and a receptacle portion. The plug portion includes a plug body, a plurality of holes formed in the plug body, a plurality of electrically-conductive connector pins recessed within the plurality of holes, and a poka-yoke structure. The receptacle portion includes a receptacle body, a plurality of electrically-insulative hollow posts extending from the receptacle body, a plurality of electrically-conductive connector pins, each being partially disposed within and partially disposed outside of a respective hollow post, and a complementary poka-yoke structure. The poka-yoke structures cooperate to facilitate proper alignment of the electrically-conductive connector pins when the receptacle portion is connected to the plug portion.

### CROSS-REFERENCE TO RELATED APPLICATIONS

This application claims the benefit of U.S. provisional application No. 63/324,352, filed 28 Mar. 2022, which is hereby incorporated by reference as though fully set forth herein.

### BACKGROUND

The present disclosure relates generally to electrophysiology catheters. In particular, the present disclosure relates to an electrical connector such as may be used to connect an electrophysiology catheter, for example a pulsed field ablation (PFA) catheter, to system electronics (e.g., high voltage/high current sources).

Ablation therapy may be used to treat various conditions afflicting the human anatomy. One such condition in which ablation therapy may be used is the treatment of cardiac arrhythmias. When tissue is ablated, or at least subjected to ablative energy generated by an ablation generator and delivered by an ablation catheter, lesions form in the tissue. Electrodes mounted on or in ablation catheters are used to create tissue necrosis or apoptosis in cardiac tissue to correct conditions such as atrial arrhythmia (including, but not limited to, ectopic atrial tachycardia, atrial fibrillation, and atrial flutter). Arrhythmias can create a variety of dangerous conditions including loss of synchronous atrioventricular contractions and stasis of blood flow. It is believed that the primary cause of atrial arrhythmias is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that may lead to arrhythmias.

Electroporation is a non-thermal ablation technique that involves applying strong electric fields that induce pore formation in the cellular membrane. The electric field may be induced by applying a relatively short duration pulse which may last, for example, from a nanosecond to several milliseconds. Such a pulse may be repeated to form a pulse train. When such an electric field is applied to tissue in an in vivo setting, the cells in the tissue are subjected to a trans-membrane potential, which opens the pores on the cell wall. Electroporation may be reversible (i.e., the temporarily-opened pores will reseal) or irreversible (i.e., the pores will remain open, causing cellular destruction). For example, in the field of gene therapy, reversible electroporation is used to transfect high molecular weight therapeutic vectors into the cells.

In other therapeutic applications, a suitably configured pulse train alone may be used to cause cell destruction, for instance by causing irreversible electroporation (IRE). This is known as pulsed field ablation (PFA).

The electrodes used for electroporation therapy may be powered either collectively, in groups, or individually. To fire electrodes either individually or in groups, however, requires that the electrodes be isolated from each other, such that the firing electrode(s) can be maintained at the appropriate high voltage while surrounding electrodes remain at zero volts. Indeed, from both a performance standpoint and a safety standpoint, both creepage and clearance isolation should be present.

Extant electrical connectors, however, either provide isolation between groups of electrodes or no isolation at all. Without isolation, the therapy applied may be less effective (e.g., because the high voltage intended to be applied by a single electrode is instead reduced and spread over multiple electrodes).

Electrical isolation should also exist between electrical components and the practitioner, who will be holding and manipulating the catheter during a procedure. This may be particularly so at the point of connection between the catheter and system electronics (e.g., high voltage/high current sources). To promote this isolation, it is desirable that the connection between the catheter and system electronics be water resistant, preventing the infiltration of fluids (e.g., saline) into the connection.

### BRIEF SUMMARY

Disclosed herein is an electrical connector for a catheter. The electrical connector for the catheter includes: a cylindrical receptacle body; a plurality of electrically-conductive connector pins disposed at least partially within the receptacle body, the plurality of electrically-conductive connector pins including a set of positive polarity connector pins and a set of negative-polarity connector pins; a flexible electronic circuit secured to the receptacle body and including a central portion; and a plurality of leaves extending from the central portion and spaced at intervals about a perimeter of the central portion. The plurality of leaves includes: a first leaf including a first plurality of conductive contact pads and a first plurality of conductive traces conductively coupling the first plurality of conductive contact pads to the set of positive polarity connector pins; and a second leaf including a second plurality of conductive contact pads and a second plurality of conductive traces conductively coupling the second plurality of conductive contact pads to the set of negative polarity connector pins. The first leaf and the second leaf extend from opposing sides of the central portion.

In embodiments of the disclosure, the plurality of electrically-conducive connector pins further includes at least one signal pin and the flexible electronic circuit further includes a third leaf extending from the central portion, the third leaf including at least one conductive contact pad and at least one conductive trace conductively coupling the at least one conductive contact pad to the at least one signal pin.

The central portion of the flexible electronic circuit includes a region of interface between the flexible electronic circuit and the plurality of electrically-conductive pins. The electrical connector can include an encapsulant, such as an epoxy, surrounding the plurality of electrically-conductive connector pins within the region of interface.

It is also contemplated that the plurality of leaves can be encapsulated within an encapsulant, such as a hot melt adhesive, an epoxy, a silicone, and/or a thermoplastic potting/molding compound.

A constraint, such as an o-ring, may surround the plurality of leaves within the encapsulant.

According to aspects of the instant disclosure, the encapsulant further encapsulates at least a portion of the cylindrical plug body, which minimizes the possibility that the encapsulant will separate from the cylindrical plug body under axial loading. In particular embodiments, the cylindrical receptacle body can include a mechanical locking structure to help facilitate this advantage.

It is desirable for the first plurality of conductive traces to be disposed within a single layer of the first leaf and for the second plurality of conductive traces to be disposed within a single layer of the second leaf.

It is also desirable to include a poka-yoke structure, such as a D-shaped post, disposed at least partially within the receptacle body. A height of the poka-yoke structure can exceed a height of the plurality of electrically-conductive pins.

In still other embodiments of the disclosure, the cylindrical receptacle body includes: at least one retention window extending from an outer surface of the receptacle body to an inner surface of the receptacle body; a layer of tape on the outer surface of the cylindrical receptacle body covering the at least one retention window; and a layer of heat shrink on the outer surface of the cylindrical receptacle body covering the layer of tape.

Also disclosed herein is an electrical connector for a catheter cable, including: a cylindrical plug body; a plurality of electrically-conductive connector pins disposed at least partially within the plug body; and a first water-resistant seal disposed about a perimeter of the cylindrical plug body.

The electrical connector can further include a cable coupled to a proximal end of the plug body and a second water-resistant seal surrounding an interface between the cable and the proximal end of the plug body.

The first water-resistant seal can include a ring having an upright inner wall and a beveled outer wall, with the upright inner wall and the beveled outer wall defining a trench therebetween that extends about a perimeter of the first water-resistant seal. This first water-resistant seal can be oriented such that an opening of the trench faces towards a proximal end of the plug body.

A poka-yoke structure, such as a D-shaped post, may be disposed at least partially within the plug body. A height of the poka-yoke structure can exceed a height of the plurality of electrically-conductive pins.

The instant disclosure also provides an electrical connector for a catheter, including a receptacle portion and a plug portion. The receptacle portion includes: a cylindrical receptacle body; a plurality of hollow posts extending from the cylindrical receptacle body, wherein the plurality of hollow posts are electrically insulative; a first plurality of electrically-conductive connector pins, wherein a first portion of each electrically-conductive connector pin of the first plurality of electrically-conductive connector pins is disposed within a respective hollow post of the plurality of hollow posts and a second portion of each electrically-conductive connector pin of the first plurality of electrically-conductive connector pins extends out of the respective hollow post of the plurality of hollow posts; and a first poka-yoke structure. The plug portion includes: a cylindrical plug body; a plurality of holes formed in the cylindrical plug body; a second plurality of electrically-conductive connector pins recessed within the plurality of holes; and a second poka-yoke structure complementary to the first poka-yoke structure. The first poka-yoke structure and the second poka-yoke structure cooperate to facilitate proper alignment of the first plurality of electrically-conductive connector pins and the second plurality of electrically-conductive connector pins when the receptacle portion is connected to the plug portion.

For example, the first poka-yoke structure can include a D-shaped post and the second poka-yoke structure can include a D-shaped receptacle. Still further, a height of the first poka-yoke structure can exceed a height of the first plurality of electrically-conductive connector pins.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1A is a diagrammatic and block diagram view of an illustrative system for electroporation therapy.

FIG. 1B is a schematic representation of a catheter for use in connection with the illustrative electroporation system of FIG. 1A.

FIG. 2 depicts a receptacle portion of an electrical connector according to embodiments of the instant disclosure.

FIG. 3A is a top view of the receptacle portion shown in FIG. 2.

FIG. 3B is a front view of the receptacle portion shown in FIG. 2.

FIG. 3C is a side view of the receptacle portion shown in FIG. 2.

FIG. 3D is a cross-section of the receptacle portion taken along line D-D in FIG. 3B.

FIG. 4 illustrates a flexible electronic circuit as used in connection with an electrical connector according to aspects of the instant disclosure.

FIGS. 5A and 5B illustrate encapsulation of a flexible electronic circuit according to certain embodiments disclosed herein.

FIG. 6 depicts a plug portion of an electrical connector according to embodiments of the instant disclosure.

FIG. 7A is a top view of the plug portion shown in FIG. 6.

FIG. 7B is a front view of the plug portion shown in FIG. 6.

FIG. 7C is a side view of the plug portion shown in FIG. 6.

FIG. 7D is a cross-section of the plug portion taken along line D-D in FIG. 7B.

FIG. 8 illustrates an embodiment of a water-resistant seal suitable for use with an electrical connector as disclosed herein.

FIG. 9 depicts a receptacle portion, such as shown in FIG. 2, mated to a plug portion, such as shown in FIG. 6.

FIGS. 11A and 11B depict an exemplary poka-yoke structure to facilitate proper alignment in an electrical connector according to aspects of the instant disclosure.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

Aspects of the instant disclosure relate to electrical connectors. Although embodiments of the disclosure will be described in connection with electrical connectors utilized to connect electrophysiology catheters to associated electronics in an electroporation therapy system (e.g., a pulsed field ablation system), those of ordinary skill in the art will appreciate that the teachings herein can be applied to good advantage in other contexts as well.

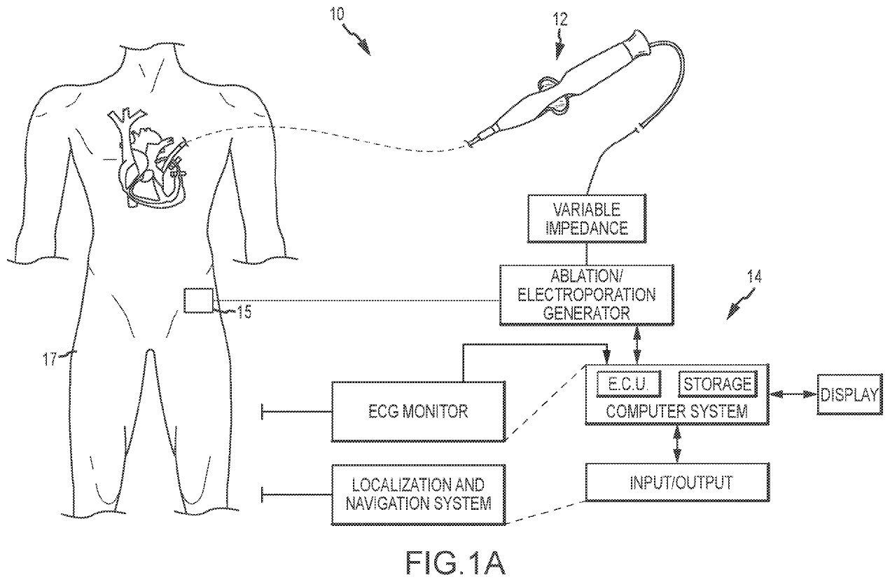

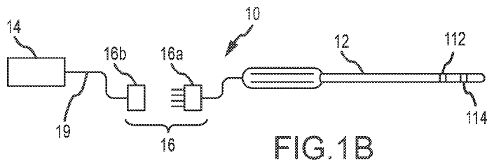

FIG. 1A is a diagrammatic and block diagram view of a system 10 for electroporation (e.g., pulsed field ablation) therapy. In general, the various embodiments include an electrode assembly disposed at the distal end of a catheter 12. As used herein, “proximal” refers to a direction toward the end of the catheter near the clinician, and “distal” refers to a direction away from the clinician and (generally) inside the body of a patient 17. The electrode assembly includes one or more individual, electrically-isolated electrode elements. Each electrode element, also referred to herein as a catheter electrode, is individually wired such that it can be selectively paired or combined with any other electrode element to act as a bipolar or multi-polar electrode.

System 10 may be used for irreversible electroporation to destroy tissue. In particular, system 10 may be used for electroporation-induced primary apoptosis therapy, which refers to the effects of applying electric fields in such manner as to directly cause an irreversible loss of plasma membrane (cell wall) integrity leading to its breakdown and cell apoptosis. This mechanism of cell death may be viewed as an “outside-in” process, meaning that the disruption of the outside wall of the cell causes detrimental effects to the inside of the cell. Typically, for classical plasma membrane electroporation, electric current is delivered as a pulsed electric field (i.e., pulsed field ablation (PFA)) in the form of short-duration pulses (e.g., about 0.1 ms to about 20 ms duration) between closely-spaced electrodes capable of delivering an electric field strength of about 0.1 kV/cm to about 1.0 kV/cm. As described, for example, in international application publication WO2019173309A1, which is hereby incorporated by reference as though fully set forth herein, system 10 may be used with a catheter for high output (e.g., high voltage and/or high current) electroporation procedures.

FIG. 1B schematically illustrates catheter 12 as connected to electronics 14 within system 10. As those of ordinary skill in the art will appreciate, and as shown in FIG. 1A, electronics 14 may include an ablation/electroporation generator, an electroanatomical mapping system, a computer system, a display, and the like.

Catheter 12 may also include thereon one or more electrodes 112, 114 (collectively referred to herein as an “electrode assembly”), which may be used for a variety of diagnostic and/or therapeutic purposes including, without limitation, cardiac mapping and/or electroporation therapy (e.g., pulsed field ablation). For example, and in some embodiments, the electrode assembly may be configured as a bipolar electrode assembly for use in bipolar-based electroporation therapy. Specifically, electrodes 112, 114 may be individually electrically coupled to generator 14 (e.g., via suitable electrical wire or other suitable electrical conductors connected through electrical connector 16 as discussed in further detail herein) and configured to be selectively energized (e.g., by an electroporation generator 14 and/or an associated computer system) with opposite polarities to generate a potential and corresponding electric field therebetween for IRE therapy. That is, one of electrodes 112, 114 can be configured to function as a cathode, and the other can be configured to function as an anode for a given therapy segment.

Electrodes 112, 114 may be any suitable electroporation electrodes. In an exemplary embodiment, electrodes 112, 114 are ring electrodes, though electrodes 112, 114 may have any other shape or configuration. Those of ordinary skill in the art will recognize that the shape, size, and/or configuration of electrodes 112, 114 may impact various parameters of the applied electroporation therapy. For example, increasing the surface area of one or both electrodes 112, 114 would decrease impedance, in turn decreasing the current that would need to be applied in order to achieve the voltage level required to cause tissue destruction.

Moreover, although each of electrode 112 and electrode 114 are illustrated as single electrodes, either or both of electrode 112 and electrode 114 may be alternatively embodied as two or more discrete electrodes.

Further, while the electrode assembly is described as a bipolar electrode assembly, it should be understood that in some embodiments, the electrode assembly may be configured as a unipolar electrode assembly and use a patch electrode on the patient's skin (e.g., 15) as a return or indifferent electrode.

Also shown in FIG. 1B is an electrical connector 16, including a receptacle portion 16a and a plug portion 16b, aspects of which are described in detail below. Additional aspects of receptacle portion 16a and plug portion 16b are disclosed in International Application No. PCT/US21/57176, which is hereby incorporated by reference as though fully set forth herein.

As shown, receptacle portion 16a is connected to catheter 12, while plug portion 16b is connected to electronics 14 via cable 19, but this arrangement could be reversed without departing from the scope of the instant disclosure. The ordinarily skilled artisan will appreciate that, when receptacle portion 16a is mated to plug portion 16b, catheter 12 becomes electrically coupled to electronics 14, enabling power, data, and other electrical signals to pass between the two. In particular, and as mentioned above, electrical connector 16 permits each individual electrode on catheter 12 to be individually and selectively paired or combined with any other electrode (or electrodes) to act as a bipolar or multi-polar electrode. Likewise, as described in further detail below, electrical connector 16, including the connection between receptacle and plug portions 16a, 16b, is water-resistant.

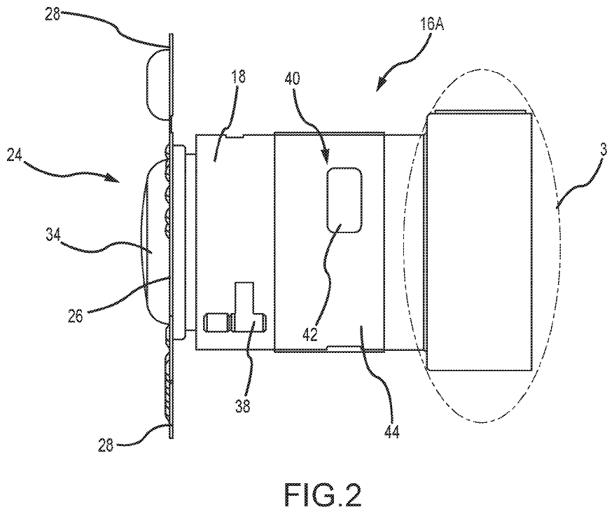

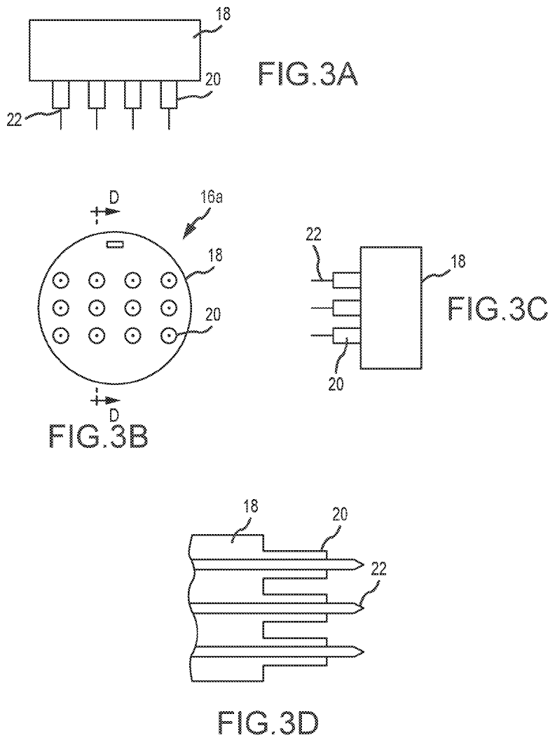

FIG. 2 depicts an embodiment of receptacle portion 16a. FIGS. 3A, 3B, and 3C are, respectively, top, front, and side views of region 3 of receptacle portion 16a as labeled in FIG. 2. FIG. 3D is a cross-sectional view taken along line D-D in FIG. 3B.

As shown to good advantage in FIGS. 2 and 3A-3D, receptacle portion 16a includes a generally cylindrical receptacle body 18 and a plurality of hollow posts 20 extending therefrom. Other shapes for receptacle body 18 are contemplated and regarded as within the scope of the instant disclosure.

In embodiments of the disclosure, hollow posts 20 are integrally formed with receptacle body 18, such as by molding receptacle body 18 and hollow posts 20 as a unitary assembly. Further, hollow posts 20 are electrically insulative. Receptacle body 18 may also be electrically insulative.

A corresponding plurality of electrically-conductive connector pins 22 extend from receptacle portion 16a. More particularly, a first portion of each connector pin 22 is disposed within a hollow post 20, while a second portion of each connector pin 22 extends out of the hollow post 20.

FIGS. 3A-3D depict a total of twelve hollow posts 20 and corresponding connector pins 22. It should be understood, however, that this configuration is merely exemplary and that any number of hollow posts 20 and connector pins 22, in any arrangement, are contemplated within the instant disclosure depending upon the specific application and/or needs associated with catheter 12 and/or electronics 14.

As those of ordinary skill in the art will appreciate, connector pins 20 form part of the electrical connection between electronics 14 and, for example, electrodes on catheter 12 (such as 112, 114, and the like). A certain subset of connector pins 20 will therefore be coupled to electrodes that function as anodes; this subset will be referred to herein as a set of positive polarity connector pins. Likewise, another subset of connector pins 20 will be coupled to electrodes that function as cathodes; this subset will be referred to herein as a set of negative polarity connector pins.

Still other connector pins 20 may be coupled to additional electronic components within catheter 12, such as temperature sensors, sensors (e.g., magnetic coil sensors) for localizing catheter 12 with an electroanatomical mapping system, data storage devices (e.g., EEPROMs), analog/digital mux chips, and the like. Such connector pins 20 are referred to herein as “signal pins.”

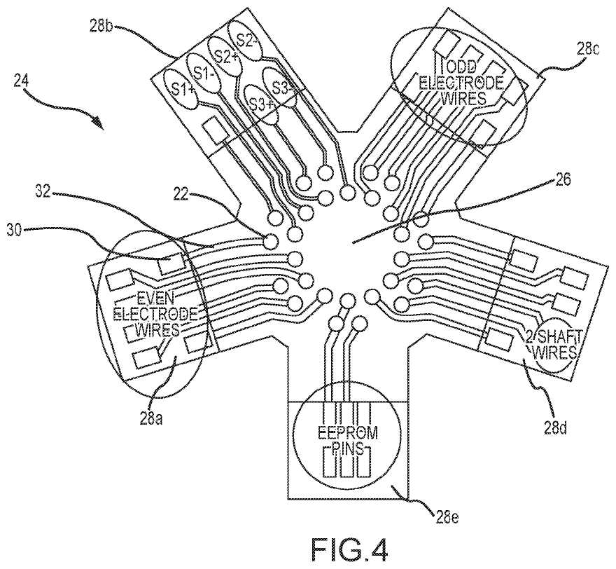

FIG. 2 further illustrates a flexible electronic circuit 24 that is secured to receptacle body 18. Referring to the more detailed depiction of flexible electronic circuit 24 in FIG. 4, it is shown that flexible electronic circuit 24 generally includes a central portion 26 and a plurality of leaves 28 extending from central portion 26 and spaced at intervals (and, in some embodiments, at generally even intervals) about a perimeter of central portion 26. Although FIG. 4 depicts a total of five leaves 28a-28e, this is merely exemplary, and those of ordinary skill in the art will appreciate that any number of leaves 28 can be utilized without departing from the instant teachings.

The general construction of each leaf 28 is substantially identical and includes a plurality of conductive contact pads 30 and a plurality of traces 32 that conductively couple conductive contact pads 30 to connector pins 22. In the interest of clarity, however, only a single contact pad 30, trace 32, and connector pin 22 is labeled in FIG. 4. As those of ordinary skill in the art will appreciate, contact pads 30 provide solder points for interconnection with wiring that can extend through catheter 12 to, for example, electrodes 112, 114 thereon.

Desirably, one leaf (28a) is dedicated for connection to the set of positive polarity connector pins 22, and a second leaf (28c) is dedicated for connection to the set of negative polarity connector pins 22. Additional leaves (28b, 28d, 28e) can be dedicated for connection to signal pins.

Moreover, leaves 28a, 28c respectively dedicated for connection to the set of positive polarity connector pins 22 and the set of negative polarity connector pins 22 extend from substantially opposing sides of central portion 26 (though they need not be precisely diametrically-opposed to each other). This spacing advantageously increases electrical isolation between the electrodes operating as anodes and those operating as cathodes and their respective connector pins 22, and thus minimizes the risk of electrical shorts within receptacle portion 16a.

In an additional aspect of the disclosure, the layout of traces 32 on each leaf 28 is designed such that all traces 32 can be disposed within a single layer of leaf 28. A single trace layer configuration minimizes the thickness and maximizes the flexibility of leaf 28, which in turn simplifies manufacture of receptacle portion 16a. Indeed, it is desirable that each leaf 28 is thin enough to avoid compromising its flexibility, but thick enough to avoid compromising its structural integrity when flexed into position during the manufacturing process as described in further detail below. In certain embodiments of the disclosure, leaves 28 are between about 0.003″ and about 0.006″ thick; in particular embodiments of the disclosure, leaves 28 are about 0.0035″ thick.

In yet another aspect of the disclosure, each trace 32 is wide enough to minimize its electrical resistance, and thus the amount of heat it generates, when relatively high current (between about 6 A and about 20 A) is flowing therethrough. In certain embodiments of the disclosure, traces 32 may have a cross-sectional area at least as large as the gauge size of the wires extending from conductive contact pads 30 to catheter electronics. For example, in certain aspects of the disclosure, traces 32 can be about 0.0014″ thick and at least about 0.018″ wide.

As depicted in FIG. 4, central portion 26 of flexible electronic circuit 24 includes a region of interface with connector pins 22. To increase electrical isolation between connector pins 22 where they interface with flexible electronic circuit 24, aspects of the instant disclosure include an encapsulant 34 (visible in FIG. 2) surrounding connector pins 22. Suitable encapsulants include, without limitation, epoxies, RTV silicone, two part silicone, thermoplastic potting/molding compounds, and the like. Epoxy may be a particularly desirable material for encapsulant 34, insofar as it is easily inspected for voids and/or bubbles that may otherwise compromise electrical isolation between connector pins 22. The use of encapsulant 34 also allows receptacle portion 16a to be electrically tested with relatively high voltages (e.g., higher than about 2000 V), prior to assembly to catheter 12. This minimizes waste during manufacture, as it allows for the identification of defects within receptacle portion 16a, prior to its connection to catheter 12, and thus minimizing the risk of loss of catheter 12 to a defect within receptacle portion 16a.

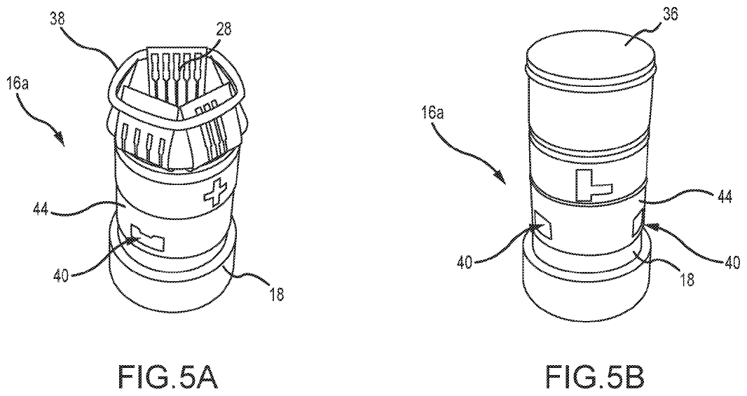

FIGS. 5A and 5B illustrate additional aspects of the manufacture of receptacle portion 16a. In particular, FIG. 5A illustrates that leaves 28 can be flexed into a generally cylindrical shape prior to securing receptacle portion 16a to catheter 12. Leaves 28 can then be encapsulated within an encapsulant 36, as shown in FIG. 5B, in order to fix them in the flexed configuration. Suitable materials for encapsulant 36 include, without limitation, hot melt adhesives, epoxies, RTV silicones, two-part silicones, and thermoplastic potting/molding compounds. Encapsulant 36 contributes to the electrical isolation of flexible electronic circuit 24; it also enhances water resistance.

Prior to encapsulation, an o-ring or other constraint 38 (e.g., tape, clips, or the like) may be applied to flexed leaves 28. Constraint 38 holds flexed leaves 28 in position during encapsulation and helps ensure that leaves 28 are fully encapsulated within encapsulant 36 following encapsulation, which in turn improves water-resistance and reduces the risk that leaves 28 and/or the electrical connections thereon disadvantageously pull or tear away from encapsulant 36, either during manufacture (e.g., when receptacle portion 16a is secured to catheter 12) or use of catheter 12.

Encapsulant 36 may also encapsulate a portion of receptacle body 18. In certain embodiments of the disclosure, receptacle body 18 includes one or more locking structures 38 (see FIG. 2A) designed to create a tortuous path for encapsulant 36 to follow during the manufacturing process. Consequently, when encapsulant 36 solidifies, the risk that encapsulant 36 will pull away from receptacle body 18 under axial loading (that is, pulling encapsulant 36 distally and/or receptacle portion 16a proximally, as when receptacle portion 16a is being disengaged from plug portion 16b at the conclusion of a procedure) is diminished.

Receptacle body 18 can also include one or more retention windows 40 (see FIG. 2A) configured to receive corresponding retention clips on plug portion 16b. To prevent retention windows 40 from offering a path for fluid ingress, they can be covered with a layer of polyimide tape 42 on the outer surface of receptacle body 18. Tape 42 can in turn be covered with a layer of heat shrink 44, which helps hold tape 42 in place, particularly as receptacle portion 16a and the adhesive on tape 42 ages. Although multi-layered, the use of tape 42 and heat shrink 44 nevertheless minimizes both the profile of receptacle body 18 and its manufacturing cost.

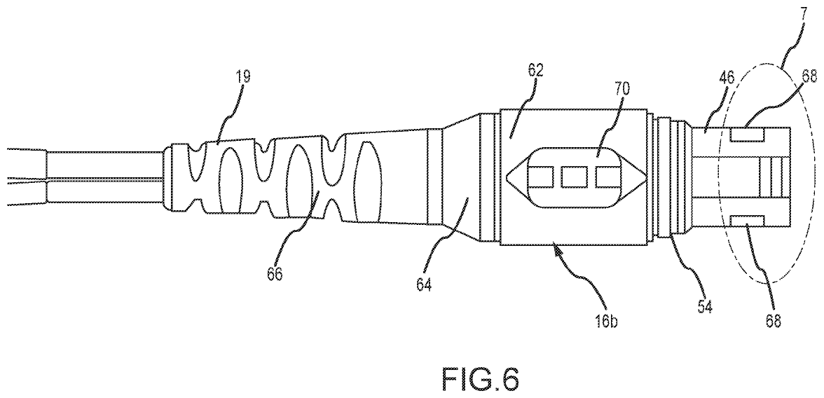

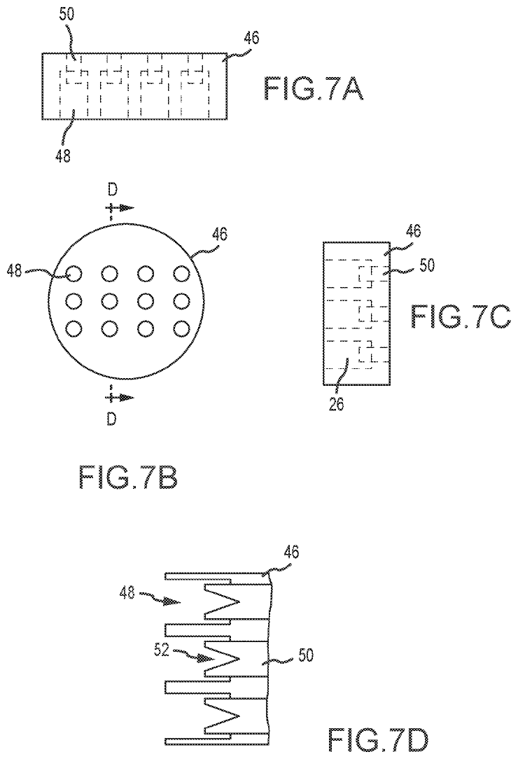

FIG. 6 depicts an embodiment of plug portion 16b. FIGS. 7A, 7B, and 7C are, respectively, top, front, and side views of region 7 of plug portion 16b as labeled in FIG. 6. FIG. 7D is a cross-sectional view taken along line D-D in FIG. 7B.

As shown to good advantage in FIGS. 6 and 7A-7D, plug portion 16b includes a generally cylindrical plug body 46 with a plurality of holes or recesses 48 formed therein. Like hollow posts 20 and receptacle body 18, plug body 46 is electrically insulative.

Within each hole 48 is an electrically-conductive connector pin 50. In embodiments of the disclosure, each connector pin 50 includes a hollow portion 52.

FIGS. 7A-7D depict a total of twelve recesses 48 and corresponding connector pins 50. It should be understood, however, that this configuration is merely exemplary and that any number of recesses 48 and connector pins 50, in any arrangement, are contemplated within the instant disclosure depending upon the specific application and/or needs associated with catheter 12 and/or electronics 14. Of course, those of ordinary skill in the art will appreciate that, because receptacle portion 16a is designed to mate to plug portion 16b, there will often be one-to-one correspondence between the configuration of posts 20 and connector pins 22 on receptacle portion 16a and the configuration of recesses 48 and connector pins 50 on plug portion 16b (that is, the two will be complementary to each other).

Returning now to FIG. 6, plug portion 16b also includes a first water-resistant seal 54 disposed about a perimeter of plug body 46. Seal 54 may be seated within a groove extending into the outer surface of plug body 46. Seal 54 facilitates a water-resistant seal between receptacle portion 16a and plug portion 16b when mated.

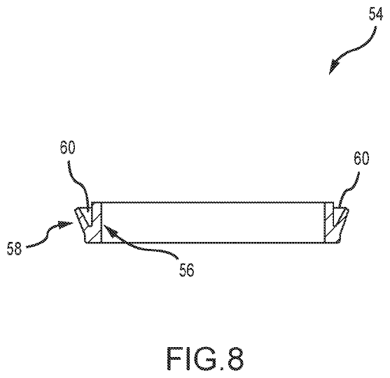

As illustrated in FIG. 8, which is a cross-section of seal 54, in embodiments of the disclosure, seal 54 is a modified o-ring having a generally upright inner wall 56 and a beveled outer wall 58. Where seal 54 is seated within a groove, the depth of the groove may be approximately equal to the thickness of inner wall 56. This configuration defines a trench 60 that extends around the perimeter of seal 54.

Seal 54 can be oriented such that trench 60 faces the proximal end 62 of plug body 46 (that is, the opening of trench 60 faces away from receptacle portion 16a). Thus, when plug portion 16b is inserted into receptacle portion 16a, beveled outer wall 58 will deflect towards upright inner wall 56, minimizing the required insertion force. Conversely, when plug portion 16b is removed from receptacle portion 16a, beveled outer wall 58 will deflect away from upright inner wall 56. This action will tend to wipe any fluid that may be present (e.g., fluid that has collected in trench 60 during a procedure and/or fluid that infiltrates between receptacle portion 16a and plug portion 16b as they are decoupled) intro trench 60 and away from electrical components (e.g., connector pins 22, 50), thereby enhancing the safety of electrical connector 16.

Referring once again to FIG. 6, a second water-resistant seal 64 can be provided between proximal end 62 of plug body 46 and cable 19. Water-resistant seal 64 forms a compression seal around plug body 46 and strain relief 66 and minimizes the risk of fluid infiltration where cable 19 meets plug body 46.

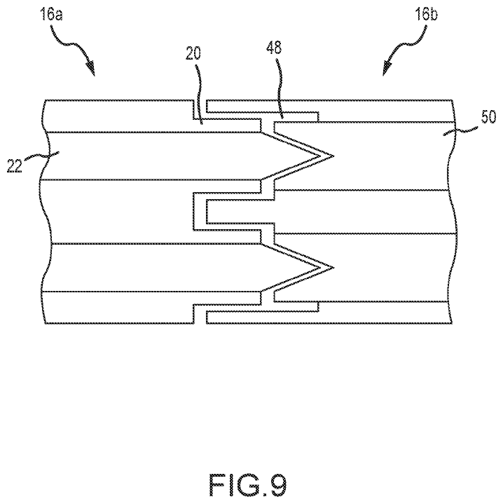

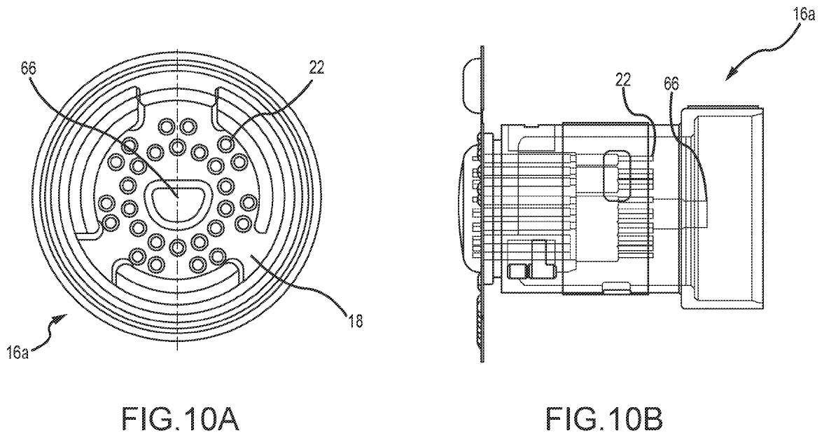

FIG. 9 illustrates a portion of electrical connector 16 when receptacle portion 16a is mated to plug portion 16b. As shown in FIG. 8, connector pins 22 of receptacle portion 16a are received into respective hollow portions 52 of connector pins 50 on plug portion 16b, thus conductively coupling connector pins 22 to connector pins 50.

FIG. 9 illustrates that posts 20 are also received within recesses 48. This advantageously increases creepage between adjacent pins; in embodiments of the disclosure, creepage may be increased by a factor of about six or more relative to extant connectors, which helps ensure electrical isolation between individual pins, in turn advantageously reducing the total number of pins required for a given number of electrodes on catheter 12.

Clearance is also advantageously increased by approximately the same factor because, once receptacle portion 16a is mated to plug portion 16b, the clearance path and creepage path are substantially the same.

Indeed, those of ordinary skill in the art will appreciate that, the further receptacle portion 16a is inserted into plug portion 16b (e.g., the further connector pins 22 of receptacle portion 16a are inserted into connector pins 50 of plug portion 16b), the greater the order of magnitude increase in both creepage and clearance.

To aid alignment between receptacle portion 16a and plug portion 16b, receptacle portion 16a and plug portion 16b can include complementary poka-yoke alignment structures at least partially within their respective bodies 18, 46. FIGS. 10A and 10B depict one suitable configuration for complementary poka-yoke alignment structures, namely, D-shaped post 66. For the sake of illustration, FIGS. 10A and 10B depict the poka-yoke alignment structure on receptacle portion 16a. Those of ordinary skill in the art will understand that plug portion 16b includes a complementary structure (e.g., a D-shaped receptacle), and will further understand that the post and receptacle sides could also be reversed (e.g., plug portion 16b could include the D-shaped post, and receptacle portion 16 could include the D-shaped receptacle). In general, however, for reasons that will become clear from the following discussion, the post side of the poka-yoke alignment structure will be co-located with the exposed electrically-conductive connector pins (e.g., connector pins 22), and the receptacle side of the poka-yoke alignment structure will be co-located with the recessed electrically-conductive connector pins (e.g., connector pins 50).

As shown in FIG. 10B, D-shaped post 66 extends further than connector pins 22; this is referred to herein as D-shaped post 66 having a greater height than connector pins 22. This configuration is advantageous because it ensures that, as plug portion 16b is inserted into receptacle portion 16a, first contact will be to D-shaped post 66 rather than connector pins 22. If receptacle and plug portions 16a, 16b are not properly rotationally aligned, they can be rotated relative to each other until they are, and the only bearing surface during such rotation will be the relatively more durable D-shaped post 66, not the relatively more fragile connector pins 22.

Electrical connector 16 can also include mating components to enhance the security of the connection between receptacle body 18 and plug body 46 when receptacle portion 16a is mated to plug portion 16b. As discussed above, receptacle body 18 includes one or more retention windows 40; plug body 46 can include a corresponding number of retention clips 68, shown in FIG. 6. To disconnect receptacle portion 16a and plug portion 16b, plug body 46 can include an actuator 70 that, when actuated, retracts retention clips 68 at least partially into plug body 46, thus disengaging them from retention windows 40.

Although several embodiments have been described above with a certain degree of particularity, those skilled in the art could make numerous alterations to the disclosed embodiments without departing from the spirit or scope of this invention.

For example, in addition to or as an alternative to the use of constraint 38, leaves 28 may include mechanical features that secure adjacent leaves 28 to each other, helping to ensure that leaves 28 are fully encapsulated within encapsulant 36.

As another example, not only can the arrangement of receptacle portion 16a and plug portion 16b be reversed (e.g., receptacle portion 16a connected to electronics 14 via cable 19 and plug portion 16b connected to catheter 12), individual features described above in connection with receptacle portion 16a could instead be applied to plug portion 16b and vice versa.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

1. An electrical connector for a catheter, comprising:
a cylindrical receptacle body;
a plurality of electrically-conductive connector pins disposed at least partially within the receptacle body, the plurality of electrically-conductive connector pins comprising a set of positive polarity connector pins and a set of negative-polarity connector pins;
a flexible electronic circuit secured to the receptacle body and comprising:
a central portion; and
a plurality of leaves extending from the central portion and spaced at intervals about a perimeter of the central portion, wherein the plurality of leaves comprises:
a first leaf including a first plurality of conductive contact pads and a first plurality of conductive traces conductively coupling the first plurality of conductive contact pads to the set of positive polarity connector pins; and
a second leaf including a second plurality of conductive contact pads and a second plurality of conductive traces conductively coupling the second plurality of conductive contact pads to the set of negative polarity connector pins,
wherein the first leaf and the second leaf extend from opposing sides of the central portion.

2. The electrical connector according to claim 1:
wherein the plurality of electrically-conducive connector pins further comprises at least one signal pin, and
wherein the flexible electronic circuit further comprises a third leaf extending from the central portion, the third leaf including at least one conductive contact pad and at least one conductive trace conductively coupling the at least one conductive contact pad to the at least one signal pin.

3. The electrical connector according to claim 1, wherein the central portion of the flexible electronic circuit comprises a region of interface between the flexible electronic circuit and the plurality of electrically-conductive pins, and wherein the electrical connector further comprises an encapsulant surrounding the plurality of electrically-conductive connector pins within the region of interface.

4. (canceled)

5. The electrical connector according to claim 1, wherein the plurality of leaves are encapsulated within an encapsulant.

6. (canceled)

7. The electrical connector according to claim 5, further comprising a constraint surrounding the plurality of leaves within the encapsulant.

8. (canceled)

9. The electrical connector according to claim 5, wherein the encapsulant further encapsulates at least a portion of the cylindrical plug body.

10. The electrical connector according to claim 9, wherein the at least a portion of the cylindrical receptacle body comprises a mechanical locking structure configured to prevent the encapsulant from separating from the cylindrical receptacle body under axial loading.

11. The electrical connector according to claim 1, wherein the first plurality of conductive traces are disposed within a single layer of the first leaf and the second plurality of conductive traces are disposed within a single layer of the second leaf.

12. The electrical connector according to claim 1, further comprising a poka-yoke structure disposed at least partially within the receptacle body.

13. The electrical connector according to claim 12, wherein the poka-yoke structure comprises a D-shaped post.

14. The electrical connector according to claim 12, wherein a height of the poka-yoke structure exceeds a height of the plurality of electrically-conductive pins.

15. The electrical connector according to claim 1, wherein the cylindrical receptacle body comprises:
at least one retention window extending from an outer surface of the receptacle body to an inner surface of the receptacle body;
a layer of tape on the outer surface of the cylindrical receptacle body covering the at least one retention window; and
a layer of heat shrink on the outer surface of the cylindrical receptacle body covering the layer of tape.

16. An electrical connector for a catheter cable comprising:
a cylindrical plug body;
a plurality of electrically-conductive connector pins disposed at least partially within the plug body; and
a first water-resistant seal disposed about a perimeter of the cylindrical plug body.

17. The electrical connector according to claim 16, further comprising:
a cable coupled to a proximal end of the plug body; and
a second water-resistant seal surrounding an interface between the cable and the proximal end of the plug body.

18. The electrical connector according to claim 16, wherein the first water-resistant seal comprises a ring having an upright inner wall and a beveled outer wall, and wherein the upright inner wall and the beveled outer wall define a trench therebetween that extends about a perimeter of the first water-resistant seal.

19. The electrical connector according to claim 18, wherein the first water-resistant seal is oriented such that an opening of the trench is towards a proximal end of the plug body.

20. The electrical connector according to 16, further comprising a poka-yoke structure disposed at least partially within the plug body.

21. The electrical connector according to claim 20, wherein the poka-yoke structure comprises a D-shaped post.

22. The electrical connector according to claim 21, wherein a height of the poka-yoke structure exceeds a height of the plurality of electrically-conductive pins.

23. An electrical connector for a catheter, comprising:
a receptacle portion comprising:
a cylindrical receptacle body;
a plurality of hollow posts extending from the cylindrical receptacle body, wherein the plurality of hollow posts are electrically insulative;
a first plurality of electrically-conductive connector pins, wherein a first portion of each electrically-conductive connector pin of the first plurality of electrically-conductive connector pins is disposed within a respective hollow post of the plurality of hollow posts and a second portion of each electrically-conductive connector pin of the first plurality of electrically-conductive connector pins extends out of the respective hollow post of the plurality of hollow posts; and
a first poka-yoke structure; and
a plug portion comprising:
a cylindrical plug body;
a plurality of holes formed in the cylindrical plug body;
a second plurality of electrically-conductive connector pins recessed within the plurality of holes; and
a second poka-yoke structure complementary to the first poka-yoke structure,
wherein the first poka-yoke structure and the second poka-yoke structure cooperate to facilitate proper alignment of the first plurality of electrically-conductive connector pins and the second plurality of electrically-conductive connector pins when the receptacle portion is connected to the plug portion.

24-25. (canceled)
